(19) United States
(12) Patent Application Publication
(10) Pub. No.: US 2008/0275466 A1
(43) Pub. Date: Nov. 6, 2008

(54) Dual cannula system and method for using same

(76) Inventor: James Grant Skakoon, St. Paul, MN (US)

Correspondence Address: MUETING, RAASCH & GEBHARDT, P.A., P.O. BOX 581336, MINNEAPOLIS, MN, 55458-1336, US

(21) Appl. No.: 11/799,179

(22) Filed: May 1, 2007

(51) Int. Cl. A61B 19/00

(52) U.S. Cl. 606/130; 604/116

(57) ABSTRACT

A cannula system and corresponding method for locating a tip of an elongate member (e.g., a catheter or other therapy delivery device) in a three-dimensional space such as a cranial cavity. One embodiment of the invention may utilize a secondary or device placement cannula that fits within a primary or guide cannula. The device placement cannula may have a length that is indexed relative to a length of a mapping member.

### TECHNICAL FIELD

The present invention relates generally to medical devices and, more particularly, to cannulas and cannula systems (e.g., for placing elongate therapy delivery devices in three-dimensional space), as well as to methods for using the same.

### BACKGROUND

Medical procedures involving access to the body via a portal are known. In one instance, access to the brain through a burr hole in the skull is used to treat a variety of medical conditions, including the relief of chronic pain (e.g., via electrical stimulation of the brain) and/or the treatment of movement disorders. These burr holes may be formed to allow implantation of various instruments, such as electrodes and catheters (e.g., a parenchymal or intracerebroventricular catheter), to treat various ailments.

Use of a catheter to deliver a therapeutic agent to the brain generally involves the insertion of the catheter into the cranial cavity and dispensing the agent at the desired location or target site. During a typical implantation procedure, an incision may be made in the scalp to expose the patient's skull. After forming the burr hole through the skull, the catheter may be inserted into the brain.

As one can appreciate, precisely locating an inserted device such as a catheter is important to ensure that the therapy (e.g., therapeutic substance) is provided to the desired target site. Depending on the particular treatment administered, a tip of the inserted device may need to be located within a narrow tolerance range relative to the target site. Even minimal variation of the tip from the target site may result in unsatisfactory therapeutic results. Accordingly, reliable methods and apparatus for locating the device are needed.

To accurately place the device and avoid unintended injury to the brain, surgeons may often use stereotactic apparatus/procedures in a process known as framed stereotaxy. One exemplary stereotactic apparatus is described in U.S. Pat. No. 4,350,159 to Gouda, which may be used to position, for example, an electrode.

In framed stereotaxy, a ring-like frame is mounted to the patient's skull by pins or screws. The ring-like frame is then used to determine a three-dimensional data set, from which coordinates for the target site may be calculated. The frame may assist in guiding surgical or other instruments (e.g., catheters and electrical leads) so that they intersect the target site.

While more than adequate for most applications, current framed stereotaxy procedures may present problems with specific implantations. For example, when implanting small devices, e.g., small diameter catheters, normal shifting of the brain (upon opening of the cranium) and potential wandering of the device tip during insertion may contribute to inaccuracies in placement. Moreover, such procedures are subject to some degree of physician variability in device placement.

### SUMMARY

The present invention may overcome these and other issues by providing various device placement systems and methodologies. For instance, in one embodiment, a cannula system for positioning a device in three-dimensional space is provided. The system includes a guide cannula having: a proximal end; and a distal end implantable within a body. An elongate mapping member may also be included. The mapping member is selectively receivable within the proximal end of the guide cannula such that a distal end of the mapping member is extendable beyond the distal end of the guide cannula. An elongate device placement cannula is also provided and selectively receivable within the guide cannula. The device placement cannula has a distal end that is also extendable beyond the distal end of the guide cannula. Further, the device placement cannula has an effective length selected to provide a predetermined length differential relative to an effective length of the mapping member.

In another embodiment, a cannula system for implanting and positioning a medical device through a portal formed in a body is provided, wherein the system includes a frame fixedly attachable to the body relative to the portal. A guide cannula is also provided and includes: a proximal end operable to extend outside the portal; and a distal end implantable within the body, wherein the guide cannula is attachable to a first portion of the frame. An elongate mapping member may also be included and selectively receivable within the proximal end of the guide cannula. The mapping member has a distal end extendable beyond the distal end of the guide cannula, and the mapping member is attachable to a second portion of the frame that is movable relative to the first portion. An elongate device placement cannula is also provided and selectively receivable within the guide cannula, the device placement cannula having a distal end also extendable beyond the distal end of the guide cannula. The device placement cannula has a predetermined effective length that is less than an effective length of the mapping member.

In yet another embodiment, a method for positioning a tip of an elongate therapy delivery device in or near a target site within a body is provided. The method includes positioning a guide cannula through a portal formed in the body, wherein a distal end of the guide cannula is located proximate the target site, and a proximal end of the guide cannula is positioned relative to a reference surface located outside the body. The method also includes inserting an elongate mapping member into the guide cannula such that a distal end of the mapping member protrudes from the distal end of the guide cannula, and securing a proximal end of the mapping member to a carrier platform that is movable relative to the reference surface. The mapping member has an effective length measured from the carrier platform to the distal end of the mapping member. The method further includes: determining a location of the target site with the mapping member; removing the mapping member from the guide cannula; and inserting an elongate device placement cannula into the guide cannula, wherein the placement cannula having an effective length selected to provide a predetermined length differential relative to an effective length of the mapping member. The method may further include: securing the placement cannula to the carrier platform; inserting the therapy delivery device into the device placement cannula; and attaching the therapy delivery device relative to the carrier platform such that a distal end of the therapy delivery device is positioned at or near the target site.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1A illustrates at least a portion of a cannula system in accordance with one embodiment of the invention, the system shown with a stereotactic frame attached to a head of a patient;

FIG. 1B is a perspective view of a drive member, e.g., microdrive, for use with the stereotactic frame of FIG. 1A;

FIG. 2 is an exploded view of the components of a cannula system in accordance with one embodiment of the invention;

FIG. 3 is a flow diagram illustrating a method of locating a therapy delivery device in accordance with one embodiment of the invention;

FIGS. 4A-4G illustrate aspects of the method described in FIG. 3, wherein: FIG. 4A illustrates insertion of a first or guide cannula through a body portal (e.g., burr hole); FIG. 4B illustrates insertion of a microelectrode into the guide cannula and attachment of the microelectrode to a carrier platform of the frame, wherein the guide cannula is shown cutaway at a distal end; FIG. 4C illustrates an enlarged view of a portion of FIG. 4B; FIG. 4D illustrates insertion of a second or device placement cannula into the guide cannula; FIG. 4E illustrates insertion of a therapy delivery device, e.g., catheter, into the placement cannula; FIG. 4F illustrates withdrawal of the guide cannula and the placement cannula from the burr hole; and FIG. 4G illustrates anchoring of the catheter and removal of the catheter from the cannulas; and

FIGS. 5A-5E illustrate a therapy delivery device, e.g., catheter, in accordance with one embodiment of the invention, wherein FIG. 5A illustrates a breakaway side elevation view; FIG. 5B illustrates an enlarged breakaway view of a first portion of the catheter; FIG. 5C illustrates an enlarged breakaway view of a second portion of the catheter; FIG. 5D is a section view taken along line 5D-5D of FIG. 5A; and FIG. 5E is section view taken along line 5E-5E of FIG. 5B.

The figures are rendered primarily for clarity and, as a result, are not necessarily drawn to scale.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

The instant invention is directed to medical systems and devices, as well as to procedures for using the same. For instance, one embodiment of the invention may be directed to a cannula system or kit for implanting a tip of a device, e.g., medical device such as a catheter or electrical stimulation lead, within a three-dimensional space. In the illustrated embodiment, exemplary systems and methods are described and illustrated in the context of implanting a brain catheter into brain tissue, e.g., through a burr hole formed in the skull of a patient. However, this is not limiting as implantation of other devices, and implantation through other body portals, are contemplated without departing from the scope of the invention.

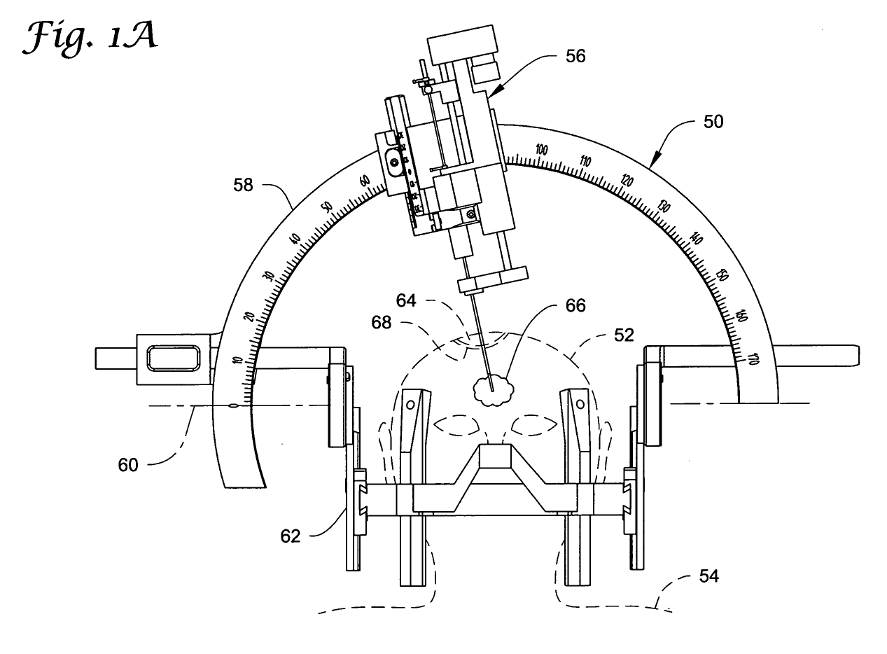

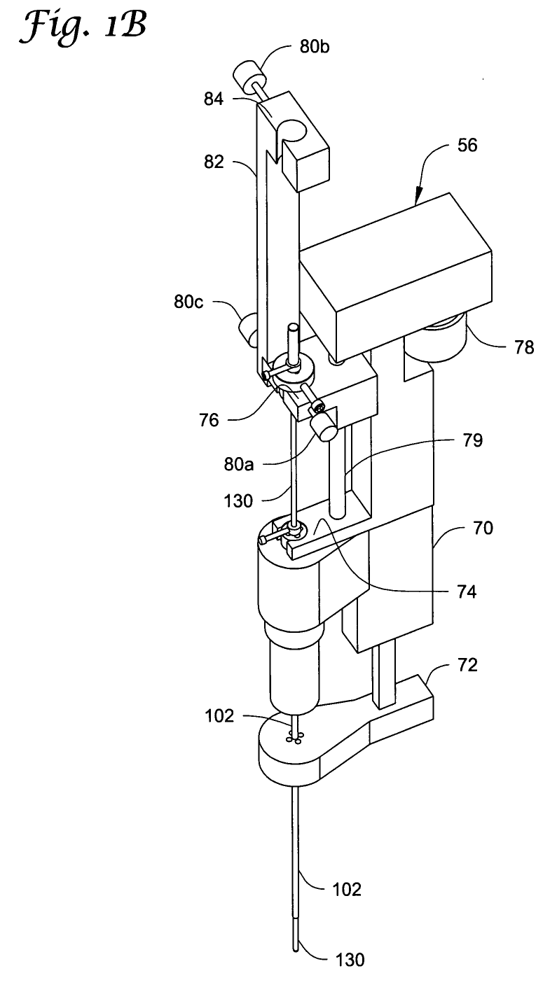

FIG. 1A illustrates an exemplary application of a cannula system in accordance with one embodiment of the present invention. As illustrated in this view, the system may be utilized with a stereotactic frame 50 as is known in the art (see, e.g., the “Leksell Stereotactic System” distributed by Elekta AB of Stockholm, Sweden). The frame 50 may be fixedly attached to a head 52 of a patient 54 relative to a burr hole 64. The frame 50 may include a drive member to which various surgical instruments may be attached. In one embodiment, the drive member may be configured as a microdrive 56 that is capable of selectively translating an elongate surgical instrument into and out of the head 52.

The frame 50 may include an arc-shaped guide 58 along which the microdrive 56 may be positioned. The arc-shaped guide 58 may also move, e.g., pivot about a transverse pivot axis 60, relative to a mounting portion 62 of the frame 50. As a result of the frame construction, the microdrive 56 may be capable of positioning the surgical instrument at most any location in localized three-dimensional space (e.g., within the head 52).

During an exemplary surgical procedure, a portal, e.g., the burr hole 64, may be formed within the skull of the patient 54. The burr hole 64 may be located based upon the previously determined approximate location of a target site 66 to which therapy is to be administered. The approximate location of the target site 66 may be determined based upon various imaging (e.g., CT, MRI) and mapping techniques as is known in the art. A burr hole anchor 68, such as that described in pending U.S. patent application Ser. No. 11/589,697, filed 30 Oct. 2006, may be used to secure a therapy delivery device or other surgical instrument, e.g., a catheter or stimulation lead, relative to the burr hole 64 after implantation.

Once the target site 66 is located and the burr hole 64 is formed, the stereotactic frame 50 may be configured such that the microdrive 56 is generally aligned with the burr hole. As a result, the microdrive may deliver the desired elongate instrument, via the burr hole, to the target site.

The microdrive 56 may, in one embodiment, be configured substantially similar to a conventional deep brain stimulation (DBS) microdrive. One exemplary microdrive is the “microTargeting Drive System for Stereotactic Positioning” distributed by FHC Inc., of Bowdoin, Me., USA and illustrated in FIG. 1B. As illustrated in this view, the microdrive may include a stationary mount 70 that attaches to the stereotactic frame 50. Extending from the mount is a guide support 72 that may be used to assist in guiding the instrument as shown. The mount 70 may define a first portion, e.g., fixed platform 74, to receive and hold an instrument in a fixed relationship to the frame 50. While described as “fixed,” the platform 74 may have some course adjustability relative to the frame.

The frame 50, e.g., microdrive 56, may also include a second portion, e.g., carrier platform 76, to receive/hold instruments. The second portion (carrier platform 76) may be selectively movable, e.g., translatable along a drive screw 79, relative to the first portion (fixed platform 74) to selectively advance or withdraw the instrument when it is attached to the carrier platform. The carrier platform 76 may be advanced or withdrawn by rotating a knob 78. Various locking thumb screws 80 (e.g., 80a, 80b, and 80c) may be provided to secure the instruments to the microdrive 50 as illustrated herein. Moreover, the microdrive 56 may include a standoff or DBS holder 82, which is attachable to the carrier platform 76. The holder 82 may also include features, e.g., holder platform 84, to receive and secure instruments as will be further described below.

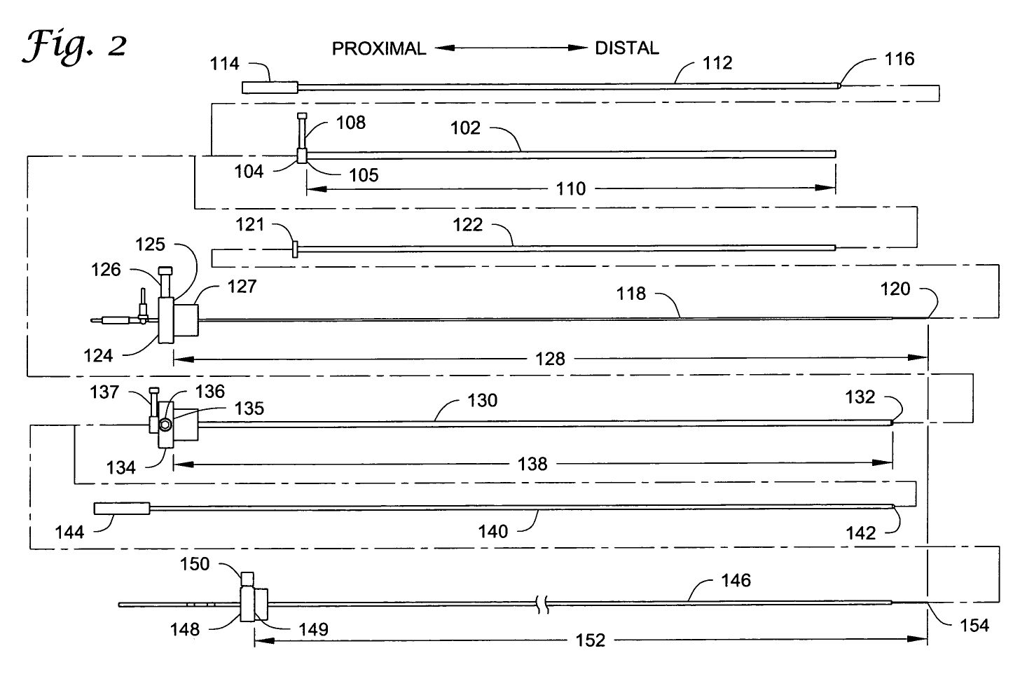

Cannula systems in accordance with embodiments of the present invention may include various components that attach to the microdrive at various locations (e.g., to the fixed or carrier platforms). A cannula system 100 in accordance with one embodiment of the invention is illustrated in FIG. 2. The system 100 may include a primary or guide cannula 102 attachable to a first portion of the frame 50 ((e.g., to the fixed platform 74 of the microdrive 56 as shown in FIG. 1B)) such that a distal end of the guide cannula is implantable within the body. The guide cannula 102 may be configured as a conventional DBS cannula and thus include a stop member 104 (e.g., formed by a flange fixed or otherwise attached) at a first (e.g., proximal) end that extends outside the burr hole 64 of the body 54 after implantation. A thumb screw 108 may thread to the stop member 104 so that other components may be secured within the guide cannula as further described below. The stop member 104, which may selectively abut the fixed platform 74 as further described below, may define an effective length 110 of the guide cannula. In one embodiment, the guide cannula 102 is a model 66-IT-03 distributed by FHC Inc., of Bowdoin, Me., USA. In an illustrative example, the guide cannula may be constructed of various materials such as stainless steel and have an outer diameter of about 1.8 millimeters, an inner or lumen diameter of about 1.6 millimeters, and an effective length 110 of about 180 millimeters, e.g., 179 millimeters. However, cannulas of other sizes and configurations are certainly possible without departing from the scope of the invention.

To assist with inserting the guide cannula 102 into tissue, a first blunt or rounded obturator or stylet 112 may also be included. A distal end 116 of the stylet 112 may be selectively inserted, via the proximal end of the guide cannula 102, into the lumen of the guide cannula until a head 114 of the stylet contacts the stop member 104. The stylet 112 may be configured such that the distal end 116 protrudes minimally from the distal end of the guide cannula 102 when the head 114 of the stylet is in contact with the stop member 104. The thumb screw 108 may then be tightened to secure the stylet 112 relative to the guide cannula 102. As a result, distal ends of the guide cannula 102 and stylet 112 may be inserted and implanted into the body with minimal coring of tissue.

As further described below, the system 100 may also include an elongate mapping member. The mapping member may be used to determine more precisely the location of the target site 66 (see, e.g., FIG. 1A). In one embodiment, the mapping member is configured as a recording microelectrode 118 having a distal end 120 that may be selectively receivable within the proximal end of the guide cannula 102 (e.g., when the stylet 112 is removed). The distal end 120 may be extendable beyond the distal end of the guide cannula as further described below. In one embodiment, the system may further include a spacer tube 122 selectively receivable within the guide cannula, e.g., within an annular gap between the inner lumen wall of the guide cannula and a cylindrical outer surface of the microelectrode 118. The spacer tube 122 may be placed within the lumen of the guide cannula 102 and secured with the thumb screw 108. The spacer tube may define an inner diameter sized to receive the mapping member, e.g., microelectrode 118, with clearance. The spacer tube may include a stop member 121 at or near its proximal end as illustrated in FIG. 2.

The microelectrode 118 may include a stop member 124 secured near its proximal end. In one embodiment, the stop member 124 is secured to the microelectrode via a thumb screw 126. The stop member 124 may be configured to seat against a second portion of the frame 50 (e.g., against the carrier platform 76 of the microdrive 56 as shown in FIG. 1B). The carrier platform 76 may include a screw 80a (see, e.g., FIG. 1B) operable to secure the stop member (a surface 127 of the stop member) relative to the carrier platform. The location of the stop member 124 may further define an effective length 128 of the microelectrode of, for example, about 250-300 millimeters, e.g., about 262.5 millimeters. In one embodiment, the microelectrode is a “microTargeting Mono- or Bi-Polar Electrode” distributed by FHC Inc., of Bowdoin, Me., USA.

The system may further include an elongate second or device placement cannula 130. The device placement cannula 130 has a distal end 132 that may be selectively receivable within the proximal end of the guide cannula 102 (e.g., when the stylet 112 and microelectrode 118/spacer tube 122 are not present) such that the distal end of the placement cannula extends beyond the distal end of the guide cannula.

The placement cannula 130 may include a stop member 134 that may secure to the placement cannula with a thumb screw 136 as shown. The stop member 134, which may abut the carrier platform 76 as further described below, may define an effective length 138 of the placement cannula. In one embodiment, the placement cannula 130 has an outer diameter of about 1.5 millimeters, an inner or lumen diameter of about 1.2 millimeters, and an effective length 138 of about 240-290 millimeters, e.g., about 252.5 millimeters.

In some embodiments, the distal end 132 of the device placement cannula 130 is configured to extend beyond the distal end of the guide cannula 102 when the components are assembled. As a result, a second obturator or stylet 140 may be provided and adapted for use with the placement cannula to reduce tissue coring. A distal end 142 of the stylet 140 may be inserted, via the proximal end of the placement cannula 130, into the lumen of the placement cannula until a head 144 of the stylet contacts the stop member 134. The stylet 140 may be configured such that the distal end 142 protrudes minimally from the distal end of the placement cannula 130 when the head 144 of the stylet is in contact with the stop member 134. A thumb screw 137 may then be tightened to secure the stylet 140 relative to the placement cannula 130.

The device placement cannula 130 may have an effective length 138 selected to provide a predetermined length differential relative to the effective length 128 of the microelectrode 118. For example, in one embodiment, the device placement cannula 130 has a predetermined effective length 138 that is less than the effective length 128 of the microelectrode, e.g., by a distance of about 10 millimeters. Such a relationship may be beneficial, as further explained below, because it may permit accurate positioning of the therapy delivery device relative to the target site 66. While described and illustrated as having an effective length 138 of a particular distance shorter than that of the mapping member (e.g., microelectrode 118), this configuration is not limiting as other embodiments may vary the length, and even the length relationship (e.g., the effective length of the placement cannula could be longer than the effective length of the microelectrode) without departing from the scope of the invention.

FIG. 2 further illustrates an exemplary therapy delivery device. In the illustrated example, the therapy delivery device is configured as a small diameter catheter 146, an embodiment of which is described in more detail below. While described and illustrated herein as a catheter, this configuration is not limiting as other therapy delivery devices, e.g., stimulation leads, and other surgical instruments are contemplated within the scope of the invention.

The catheter 146 may have a distal end 148 that may be selectively receivable within (e.g., introduced into) the proximal end of the device placement cannula 130 (e.g., when the stylet 140 is not present). The catheter 146 may have an outer cylindrical surface defined by an outer diameter sized to have a slight clearance fit with the lumen of the placement cannula 130.

As with the placement cannula 130, the catheter 146 may include a stop member 148 that may secure to the catheter with a thumb screw 150 as shown.

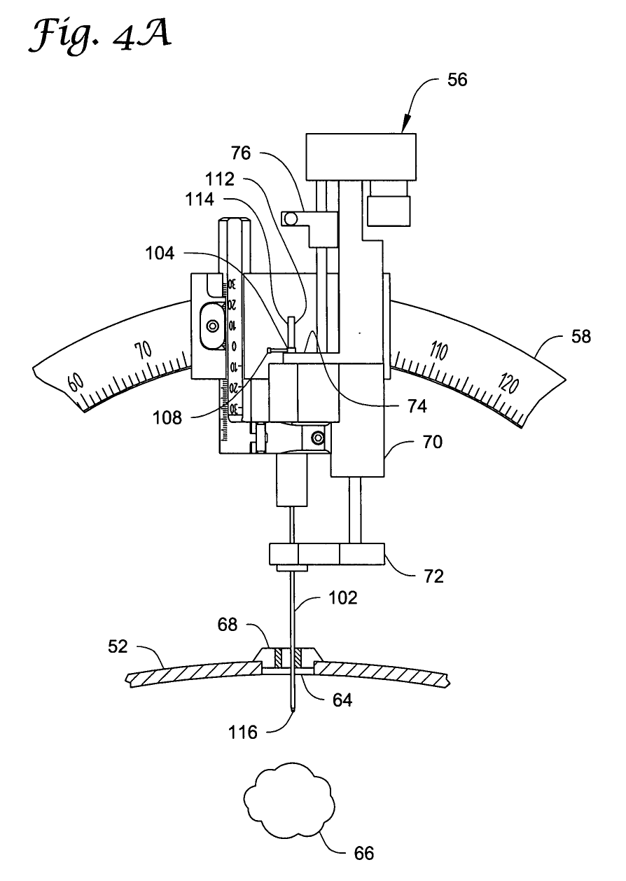

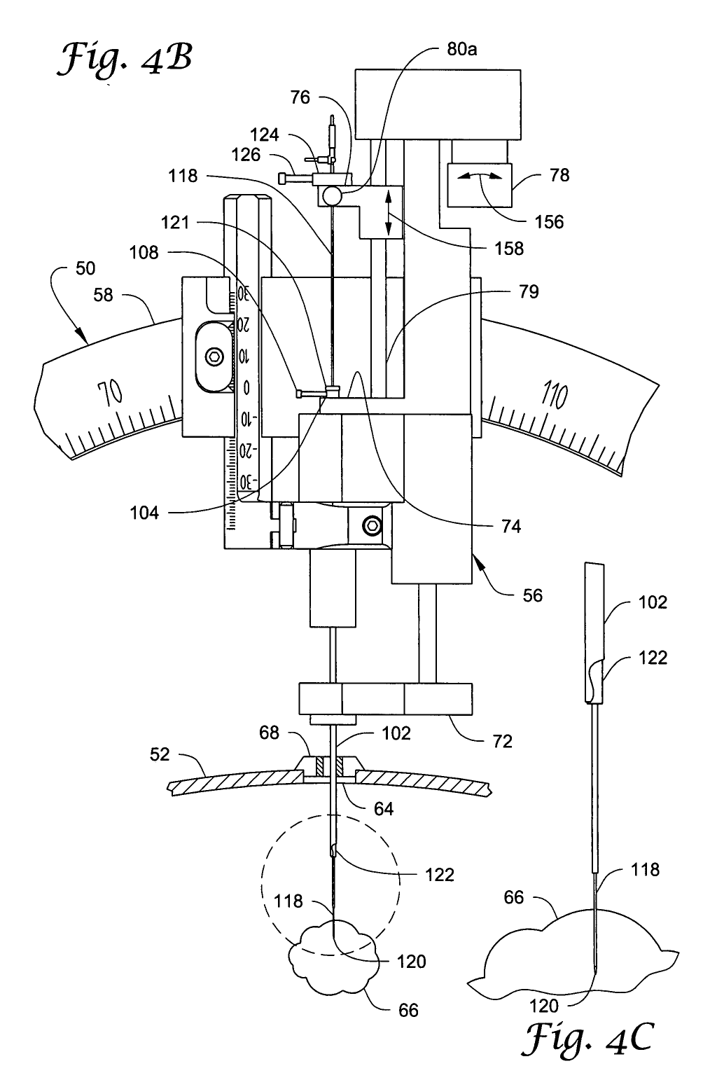

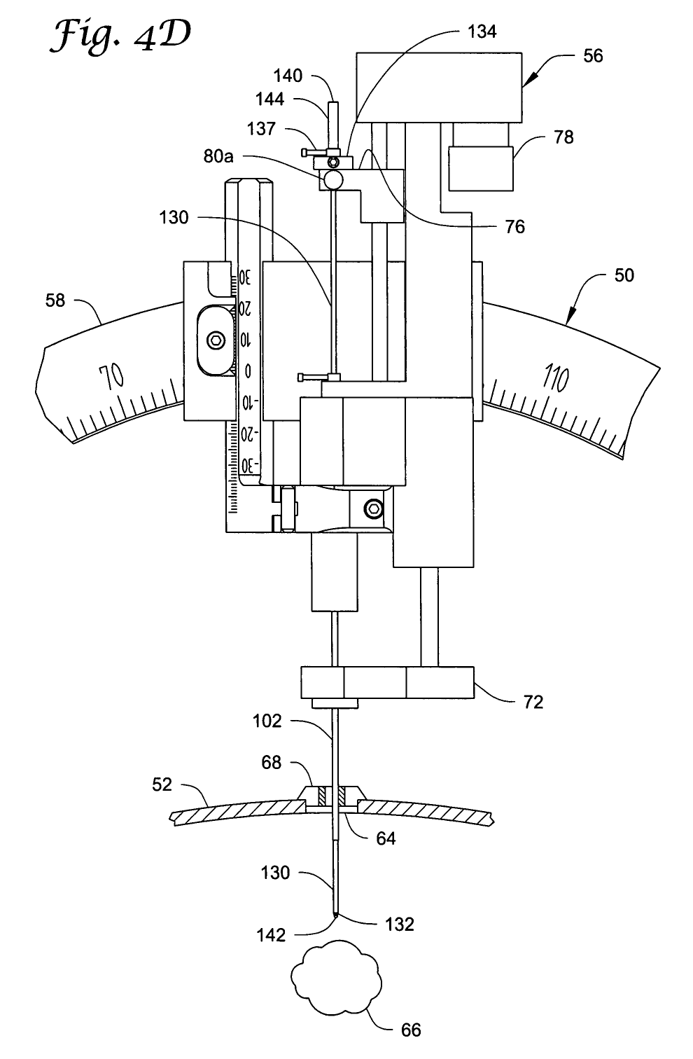

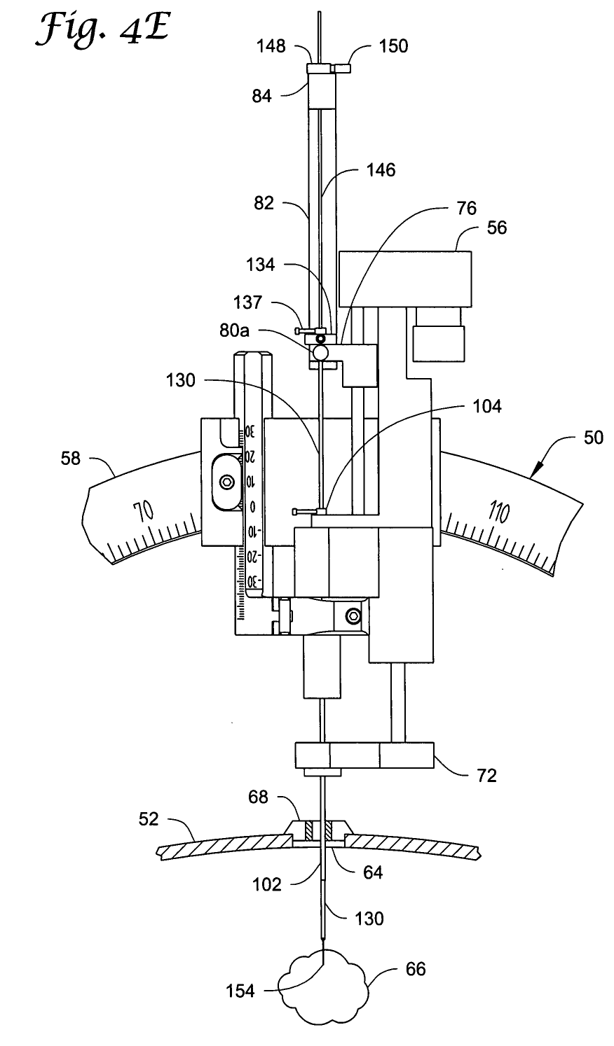

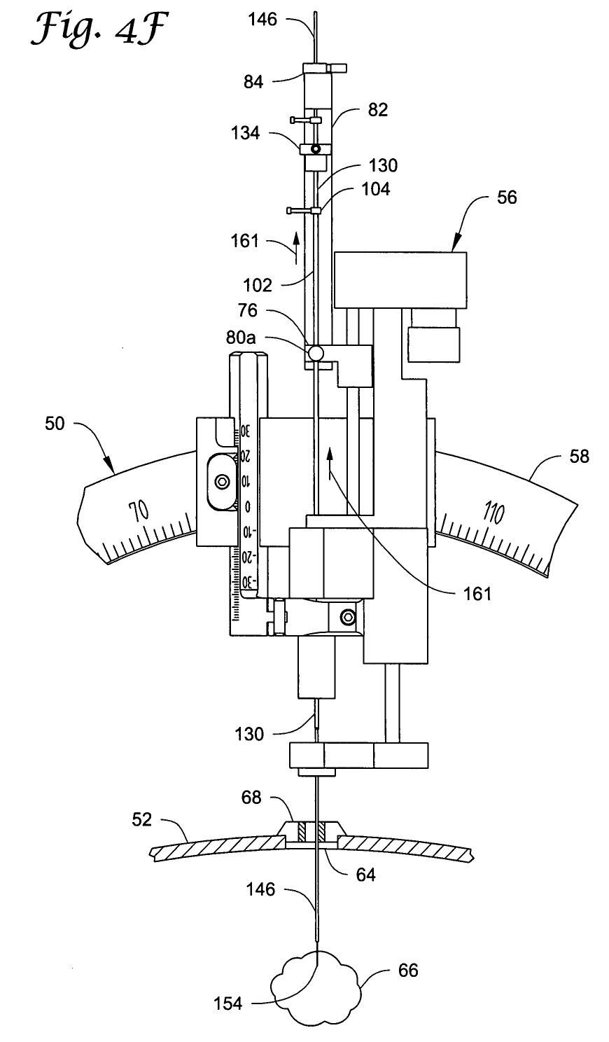

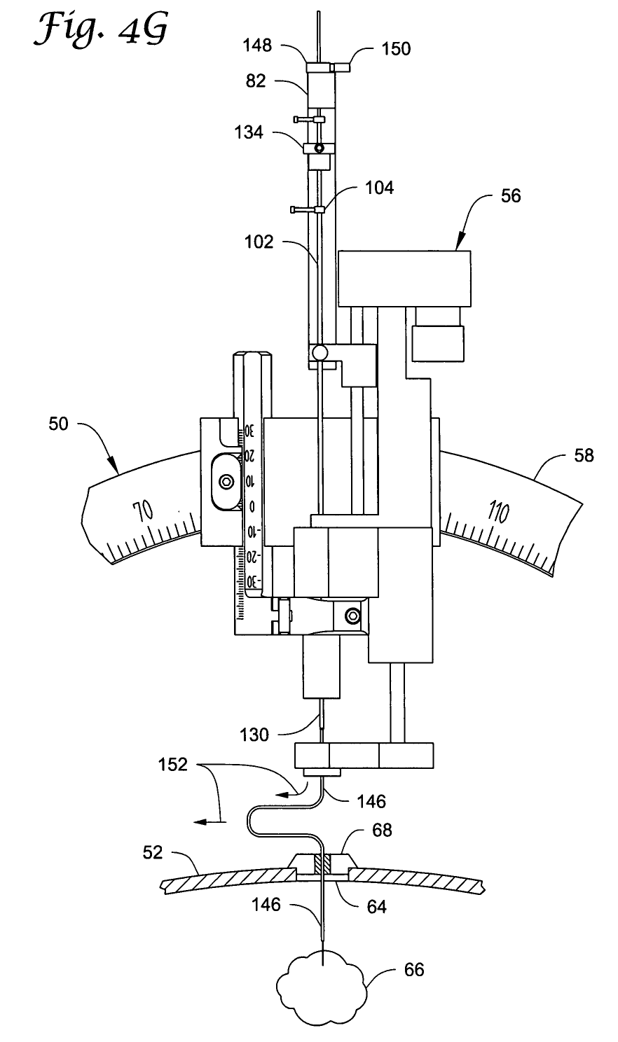

The stop member 148, which may abut the holder platform 84 (see, e.g., FIG. 1B and 4D) as further described below, may define an effective length 152 of the catheter 146. The effective length 152 may be selected to place the distal end of the catheter at a predetermined distance from the distal end of the device placement cannula as described below. In one embodiment, the catheter 146 has an outer diameter of about one millimeter, an inner or lumen diameter of about 0.1 millimeters, and an effective length 152 of about 350-450 millimeters, e.g., about 370 millimeters. The catheter may, in one embodiment, include an inner tubular member that protrudes from an outer tubular jacket by a short distance, e.g., 10 mm, at the distal end as shown in FIG. 2 and further described below.

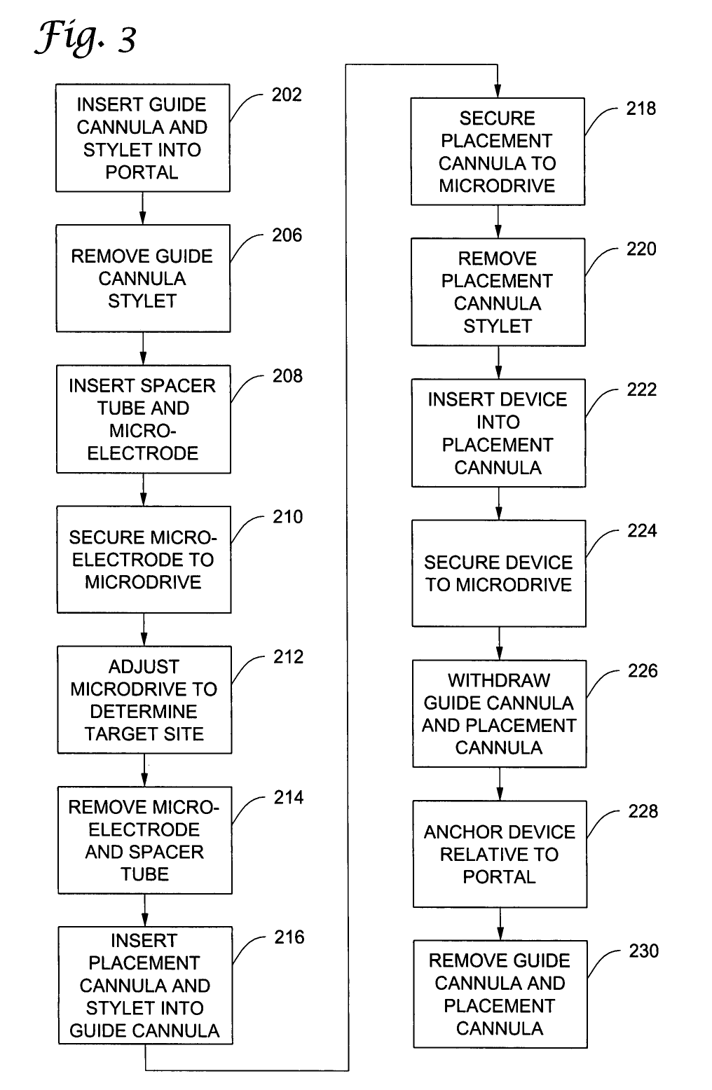

An exemplary method of using the cannula system 100, e.g., to position a tip of an elongate therapy delivery device in or near the target site within the body, will now be described with reference to FIGS. 3 and 4A-4G. FIG. 3 is a block diagram illustrating the exemplary method, while FIGS. 4A-4G illustrate various method procedures. While illustrated in a particular order herein, those of skill in the art will recognize that the processes described herein may, where feasible, occur in a different order without departing from the scope of the invention.

The illustrated procedure assumes that the stereotactic frame has been attached to the patient and positioned such that the microdrive 56 is aligned with the burr hole 64 as shown in FIG. 1A. Moreover, the burr hole anchor 68, examples of which are illustrated in, for example, U.S. patent application Ser. No. 11/589,697, may already be placed within the burr hole (although not yet configured to immobilize the catheter).

With reference to FIG. 4A, the guide cannula 102, with the stylet 112 inserted and secured (e.g., via the screw 108) therein, may be passed through (from above) the fixed platform 74 of the stationary mount 70 until a lower surface 105 (see FIG. 2) of the stop member 104 contacts an upper surface of the platform. This results in positioning of the guide cannula 102 through the portal/burr hole 64. Stated alternatively, the distal end of the guide cannula 102 and the tip 116 of the stylet may extend through the burr hole, as shown in FIG. 4A and represented at 202 in FIG. 3, and be located proximate the target site 66. The guide cannula 102 may rest upon a reference surface located outside the body, e.g., the fixed platform 74 of the microdrive 56. In alternative embodiments, the guide cannula could be physically secured to the platform 74 with a locking screw or the like that is associated with the platform. The guide cannula may be located at most any desired approach distance from the target site.

Once the guide cannula 102 is positioned on the fixed platform, the thumb screw 108 may be loosened and the stylet 112 withdrawn (e.g., upwardly through an opening in the carrier platform 76) as represented at 206 in FIG. 3. The guide cannula 102 may remain in position relative to the platform 74 as shown in FIG. 4A.

With reference to FIG. 4B, the spacer tube 122 may be inserted into the guide cannula 102 from above through the opening in the carrier platform 76 until the stop member 121 of the spacer tube contacts the proximal end (e.g., stop member 104) of guide cannula 102. The spacer tube 122 may then be secured relative to the guide cannula 102 by the thumb screw 108. The microelectrode 118 may then be inserted through the carrier platform 76 until a lower surface 125 (see FIG. 2) of the stop member 124 contacts the upper surface of the carrier platform 76 as represented at 208 in FIG. 3. At this point, the distal end 120 of the microelectrode 118 may protrude from the distal end of the guide cannula 102 as shown in FIG. 4B (FIG. 4C is an enlarged view of this portion of FIG. 4B). The proximal end of the microelectrode 118, e.g., the stop member 124, may be secured to the carrier platform 76 by a locking screw 80a as represented by 210 in FIG. 3. As a result, the effective length 128 of the microelectrode 118 may be measured from the carrier platform 76 to the distal end of the microelectrode.

The microdrive 56 may then be withdrawn and/or advanced relative to the burr hole 64 to correspondingly withdraw/advance the distal tip 120 of the microelectrode 118. The microdrive 56 may be withdrawn or advanced by rotation of the knob 78 as represented by arrows 156, which results in corresponding rotation of a screw 79. Rotation of the screw 79 causes the carrier platform 76 to move upwardly or downwardly as represented by arrows 158 in FIG. 4B.

By detecting electrical variation as the microelectrode is translated within the brain tissue, a precise location of the target site 66 may be determined as represented by 212 in FIG. 3 by detecting an electrical signal from the microelectrode tip 120. Once the target site is located, the microelectrode 118 may be loosened from the carrier platform and removed (e.g., withdrawn upwardly) from the guide cannula 102 and microdrive 56. The spacer tube 122 may similarly be loosened from the guide cannula 102, withdrawn upwardly, and removed from the microdrive 56 as represented at 214 in FIG. 3. The carrier platform 76 remains substantially in place, relative to the remaining portions of the microdrive 56, via the friction of the screw 79.

Referring now to FIG. 4D, the placement cannula 130 and accompanying stylet 140 may be secured to one another as described above and inserted into the guide cannula 102 as represented by 216 in FIG. 3. The cannula 130 and stylet 140 may be inserted downwardly through the opening in the carrier platform 76 until a lower surface 135 (see FIG. 2) of the stop member 134 contacts the upper surface of the carrier platform 76, e.g., the placement cannula is indexed from the same location as the microelectrode 118. The placement cannula 130 may then be secured relative to the carrier platform 76 by the locking screw 80a as represented by 218 in FIG. 3. Once the cannula 130 is secured relative to the carrier platform 76, the stylet 140 may be removed from the placement cannula (e.g., by first unthreading thumb screw 137) and withdrawn therefrom (e.g., upwardly in FIG. 4D) as represented by 220 in FIG. 3.

As mentioned above, the placement cannula 130 may be shorter than the length of the microelectrode 118 by a predetermined distance, e.g., 10 millimeters. As a result, the location of the distal end 132 of the placement cannula is known with a substantial degree of certainty relative to the target site 66.

As shown in FIG. 4E, the device, e.g., catheter 146, may be inserted, e.g., from above, into the placement cannula 130 as represented by 222 in FIG. 3.

The stop member 148 may have been previously positioned on the catheter 146 and attached thereto (with the thumb screw 150) using a measuring device, e.g., a DBS measuring device incorporating the DBS holder 82. The stop member 148 may further be secured to the holder platform 84 using a locking screw 80b (shown in FIG. 1B). As a result, a lower surface 149 of the stop member 148 (see FIG. 2) may abut the upper surface of the holder platform 84 as shown in FIG. 4E.

The catheter 146 may thus be secured to the DBS holder 82 (which may have previously been secured to the carrier platform 76 via a locking screw 80c (see FIG. 1B)) as represented by 224 in FIG. 3. As the effective length 152 (see FIG. 2) of the catheter 146 is selected to correspond to the effective length of the placement cannula 130, the location of the catheter distal tip 148 may be known with a substantial degree of certainty. For example, the effective length 152 of the catheter 146 may be selected to ensure that it extends only slightly from the distal end of the placement cannula 130. Once again, in the illustrated embodiment, the catheter distal end may protrude a distance of only about 10 millimeters from the distal end of the placement cannula to reach the target site. Because this distance is small, detrimental external factors such as brain movement and tip wandering are minimal, resulting in desirable placement of the device tip.

With reference now to FIG. 4F, the locking screw 80a that secures the placement cannula 130 may be loosened. Thereafter, the guide cannula 102 and placement cannula 130 may be withdrawn upwardly as represented by arrows 161 in FIG. 4F and 226 in FIG. 3. This withdrawal may result in the placement cannula approaching a lower surface of the holder platform 84. The guide cannula 102 may move upwardly and its stop member 104 may pass through the opening in the carrier platform 76. In some embodiments, the two cannulas may be moved upwardly until their respective stop members contact microdrive structure. However, complete withdrawal may not be required. Rather, only that sufficient to withdraw the distal ends of the cannulas from the burr hole 64 as shown in FIG. 4F may be necessary.

With reference to FIG. 4G, the surgeon may now manipulate the burr hole anchor 68 to anchor or secure the device, e.g., catheter 146, in place as represented by 228 in FIG. 3. The catheter may then be disconnected from the stop member 148, e.g., by loosening thumb screw 150, and pulled in the direction 152 through the microdrive 56 as shown in FIG. 4G until the catheter is separated from the frame 50. The guide cannula and placement cannula, as well as the stereotactic frame 50, may then be removed from the patient as represented by 230 in FIG. 3.

The catheter 146 may then be routed, e.g., tunneled underneath the skin or routed externally, and connected to a source containing the therapeutic agent. The skin flap (not shown) may also be sutured over the burr hole and anchor.

While configurations may certainly vary, exemplary catheters for use with the systems and methods described herein are configured such that they may be satisfactorily immobilized by the burr hole anchor 68 without occlusion of the fluid passageway. In some embodiments, the catheter may be configured as described in U.S. patent application Ser. No. ______ (Attorney docket no. 134.02740101), filed on even date herewith.

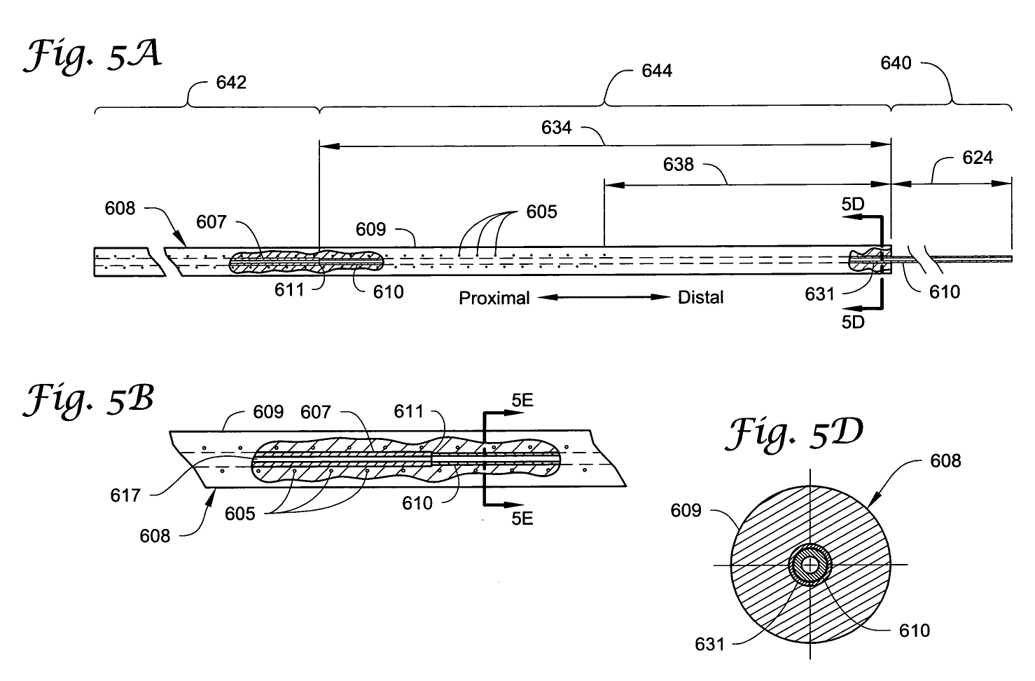

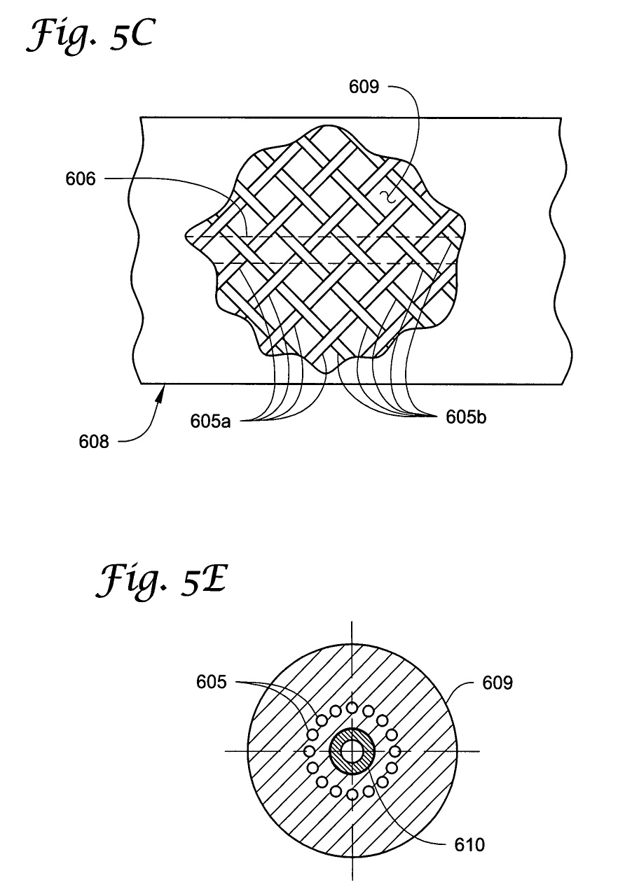

One such exemplary catheter 608 is illustrated in FIGS. 5A-5E. The catheter 608 may be similar to, and used in place of, the catheter 146 described above. It may be configured to include an elongate tubular core or core member 607 (see, e.g., FIGS. 5A and 5B) made from longitudinally flexible tubing that is resistant to compression and collapse, e.g., silica or quartz capillary tubing, or polyetheretherketone (PEEK) capillary tubing.

The core 607 may include a proximal end positioned at or near the proximal end of the catheter 608, and a distal end that terminates within the catheter body. The core 607 may also include a tubular body forming a lumen 617 spanning between the proximal and distal ends of the core. In the illustrated embodiment, the tubular core 607 may have an inner (e.g., lumen 617) diameter of about 80 micrometers to about 120 micrometers (e.g., about 100 micrometers) and an outer diameter of about 200 micrometers (e.g., about 193 micrometers), yielding a wall thickness of about 50 micrometers or less. An exemplary core 607 may be a flexible synthetic fused silica capillary having an optional thin protective polymer (e.g., polyimide) coating (forming an intermediate layer between the core and an outer covering or jacket 609) such as the TSP line of products sold by Polymicro Technologies, LLC, of Phoenix, Ariz., USA. Other embodiments may utilize a PEEK tubular core.

A flexible outer covering or jacket 609 may be formed over the tubular core 607, e.g., it may surround the tubular core and be secured or otherwise fixed relative to the core's outer surface. The jacket 609 may be formed of an elastomeric material having a radial compliance that is greater than that of the tubular core 607. In one embodiment, the elastomeric jacket 609 is made from a material selected from the group consisting of polyurethane and silicone. As a result of using a relatively compliant material, the flexible outer covering or jacket may permit high mechanical clamping/indentation forces to be applied to the catheter 608 (e.g., by the anchor 68 of FIG. 4G) to immobilize it, while the more radially rigid tubular core 607 prevents catheter occlusion under such high forces. While not limited to any particular hardness, the jacket 609 may, in one embodiment, have a hardness of about 50 to about 60 Shore D, e.g., about 55 Shore D (at the completion of manufacture).

While the flexible outer covering or jacket is described herein as an elastomeric jacket 609, this construction is not limiting as other outer covering embodiments are certainly possible without departing from the scope of the invention.

In one embodiment, the jacket 609 may have an outer diameter that is about 3 or more times larger, and preferably about 4 or more times larger (e.g., about 4 to about 6 times larger), than the outer diameter of the tubular core 607. For example, the outer diameter of the jacket 609 may be about 0.8 mm to about 1.2 mm (e.g., about 1 mm).

The catheter may optionally include one or more locator markings. For instance, a marker band, e.g., a fluoroscopic or radiopaque band 631, may be located at or near the distal end of the outer covering (e.g., jacket 609) as shown in FIGS. 5A and 5D. The band 631 may include platinum, iridium, or a similar material that may permit detection of the band with fluoroscopic or x-ray imaging. Such a configuration may be beneficial, for example, during implantation of the catheter into the body.

The catheter 608 may further optionally include other locator markings, e.g., longitudinal markings (not shown). Such longitudinal markings may be evenly spaced and include some colorant (e.g., titanium dioxide) to permit visual indication of catheter implant depth.

As illustrated in FIGS. 5A and 5B, the catheter 608 may incorporate a separate tubular tip or tip member 610, e.g., a tip member made of a material different than a material of the core member 607. The tip member 610, like the core 507, may also have proximal and distal ends as shown in FIG. 5A.

The tip 610 may be configured as a relatively rigid (both radially and longitudinally) member. For instance, in one embodiment, the tip 610 may be formed from fused silica glass tubing. In another embodiment, the tip 610 may be made from steel, e.g., type 304 stainless steel hypodermic tubing. The proximal end of the tip 610 may abut the distal end of the core 607 (e.g., be positioned in abutting contact at location 611 as illustrated in FIGS. 5A and 5B) such that a generally continuous lumen is established from the proximal end of the core 607 to the distal end of the tip 610. While described herein as abutting one another, in practice the two members 607 and 610 may have a small gap therebetween, e.g., the adjacent ends of the tip and core may be positioned to be near, rather than abut, one another. Nonetheless, the jacket 609 may effectively seal the interface and provide a generally continuous lumen as described.

While not wishing to be bound to any particular construction, the materials and geometry of the tip 510 and core 507 may be selected to produce a bending stiffness ratio (ratio of the bending stiffness of the tip to bending stiffness of the core) of about 24:1.

The jacket 609 may surround or encase longitudinal sections or portions of both the tip 610 and the core 607 as further described below. As with the catheter 146, the distal end of the tip 610 may protrude a preset distance beyond a distal end of the jacket as shown in FIG. 5A. The tip 610 may also extend into the jacket 609 a distance 634 that is sufficient to ensure retention of the tip.

FIG. 5D illustrates a section view of the distal end of the jacket 609 taken along line 5D-5D of FIG. 5A. This view (along with FIG. 5A) clearly illustrates the marker band 631 (as described above) formed, at least in one embodiment, as a ring located around the tip 610 and surrounded by the jacket 609. In one embodiment, the marker band may extend a short distance, e.g., about 1 mm, from the end of the jacket. The band 131, as well as the optional longitudinal length markings, may be visible from any radial position, e.g., they may extend 360 degrees around the catheter.

FIG. 5C illustrates a broken or cutaway view of the catheter 608. As illustrated in this view, the catheter 608 may also include strengthening members, e.g., braided members 605 helically-wound about a longitudinal length of the catheter, and/or optionally straight longitudinal members 606. For example, strengthening members may be sandwiched between the core and the flexible outer covering or jacket (e.g., such that they are surrounded by the jacket), or alternatively embedded within the jacket. Exemplary strengthening members may include polyester (e.g., polyethylene terepthalate (PET)), synthetic polymers such as Kevlar brand fiber (sold by E. I. du Pont de Nemours of Wilmington, De., USA), and liquid crystal polymers. In other embodiments, steel may be used to form the strengthening members.

In the illustrated embodiment, the strengthening members 605 may form a tubular braid located coaxially about portions of one or both of the core 607 and the tip 610 (note: the members 605 are shown only diagrammatically in the figures). Stated another way, the individual members 605 may include a plurality of first braided members 605a helically wound about at least the tubular core of the catheter 608 (e.g., about the core and tip 510) in a first or clockwise direction, and a plurality of second braided members 605b helically wound in a second, opposite or counterclockwise direction (as shown in FIG. 5C) such that individual members interweave with one another.

In one embodiment, the strengthening members 605 (e.g., the members 605a and 605b) include sixteen separate, 0.05 mm (0.002 inch) diameter PET fibers that are partially embedded within the jacket 609 as shown in FIGS. 5C and 5E, the latter of which is a section view taken along line 5E-5E of FIG. 5B. These strengthening members 605 may extend along at least a portion of the catheter 608. For example, in the embodiment illustrated in FIG. 5A, the members 605 may terminate a distance 638 short of the distal end of the jacket 609. The strengthening members 605 may also extend towards the proximal end of the catheter at least beyond the distance 634 (e.g., so that they surround the distal end of the core 607 and the proximal end of the tip 610 as shown) to increase strain relief to the catheter in the vicinity of the location 611. In the illustrated embodiment, the strengthening members 605 may extend fully to the proximal end of the catheter 608.

While not wishing to be bound to any particular embodiment, the exemplary catheter 608 may be about 400 mm (16 inches) long (including the protruding tip 610). The distance 638 (the termination offset of the strengthening members 605 from the distal end of the jacket 609) may be about 10 mm (0.4 inches), while the distance 634 at which the core member 607 abuts the tip member, may be about 20 mm (0.8 inches). As a result, the preset distance 624 may be about 10 mm (0.4 inches), which is equal to about ⅓ of the total length of the tip member 610.

The catheter embodiments illustrated in FIGS. 5A-5E may further provide a catheter having: a distal end section 640 defined by the protruding (e.g., glass or stainless steel) tip member 610; a proximal end section 642 defined by the portion of the catheter incorporating the core member 607; and a medial section 644 between the distal and proximal end sections (see, e.g., FIG. 5A). The jacket 609 may extend along and surround both the medial section 644 and the proximal section 642. The distal end section 640 may thus have a longitudinal portion with a uniform outer diameter less than an outer diameter of one or both the proximal end section 642 and the medial section 644. As a result of the different material of the core 607 and tip 610, the distal end section 640 may also have a bending stiffness that is greater than a bending stiffness of the proximal end section 642.

The outer covering or jacket 609 may be applied to the tubular core 607 and tip 610 in any known fashion. For example, it may be applied over the core 607 and tip 610 through a secondary extrusion process. Alternatively, the outer covering or jacket 609 may form a tube which slides over the tubular core 607 and tip 610 with clearance. In the case of the latter, two or more abutting tubing segments may be employed to produce the jacket 609. These multiple segments may also be beneficial in providing the proper spacing for the longitudinal markings. A shrink-wrap tube may then be placed over the assembled tubes and the entire assembly heated. Any optional strengthening members, e.g., woven fibers 605, may also be placed over the tubular core 607 or the outer covering 609 before the heat shrink tube is applied. Subsequent heating of the assembly may cause the outer covering 609 to melt and the shrink-wrap tube to constrict. Thus, the shrink-wrap tube may force the melted outer covering (and optional strengthening members) inwardly towards the tubular core 607 and bond to the same. The shrink-wrap tube may then be removed to produce the catheter 608.

Embodiments of the invention as described herein may provide systems and methods for locating an elongate member (such as a catheter or other therapy delivery device) in three-dimensional space. In some embodiments, systems are provided that include dual cannulas for locating the device, and a mapping member (such as an electrode) for determining the target site. The dual cannula embodiments may include a secondary or device placement cannula that has a length indexed to a length of the mapping member. Accordingly, precise placement of the therapy delivery device may be achieved without the variability or iterative aspects that may exist with other device placement methods.

The complete disclosures of the patents, patent applications, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations, combinations, and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A cannula system for positioning a device in three-dimensional space, the system comprising:
a guide cannula comprising: a proximal end; and a distal end implantable within a body;
an elongate mapping member selectively receivable within the proximal end of the guide cannula, the mapping member having a distal end extendable beyond the distal end of the guide cannula; and
an elongate device placement cannula selectively receivable within the guide cannula, the device placement cannula having a distal end also extendable beyond the distal end of the guide cannula, wherein the device placement cannula has an effective length selected to provide a predetermined length differential relative to an effective length of the mapping member.

2. The system of claim 1, wherein an effective length of the device placement cannula is less than an effective length of the mapping member by a distance of 10 millimeters.

3. The system of claim 1, further comprising a therapy delivery device having a distal end selectively receivable within the device placement cannula, the therapy delivery device having a length selected to place the distal end of the therapy delivery device a predetermined distance from the distal end of the device placement cannula.

4. The system of claim 3, wherein the therapy delivery device comprises a catheter.

5. A cannula system for implanting and positioning a medical device through a portal formed in a body, the system comprising:
a frame fixedly attachable to the body relative to the portal;
a guide cannula comprising: a proximal end operable to extend outside the portal; and a distal end implantable within the body, the guide cannula attachable to a first portion of the frame;
an elongate mapping member selectively receivable within the proximal end of the guide cannula, the mapping member having a distal end extendable beyond the distal end of the guide cannula, wherein the mapping member is attachable to a second portion of the frame that is movable relative to the first portion; and
an elongate device placement cannula selectively receivable within the guide cannula, the device placement cannula having a distal end also extendable beyond the distal end of the guide cannula, wherein the device placement cannula has a predetermined effective length that is less than an effective length of the mapping member.

6. The system of claim 5, further comprising a first stylet for use with positioning the guide cannula, and a second stylet for use with positioning the device placement cannula.

7. The system of claim 5, further comprising a spacer tube selectively receivable within the guide cannula, the spacer tube sized to receive the mapping member with clearance.

8. The system of claim 5, wherein the second portion of the frame comprises a carrier platform that is selectively translatable relative to the first portion of the frame.

9. The system of claim 8, wherein one or both of the mapping member and the device placement cannula are attachable to the carrier platform.

10. The system of claim 5, further comprising a therapy delivery device selectively receivable within the device placement cannula.

11. The system of claim 10, wherein the therapy delivery device comprises a catheter.

12. The system of claim 10, further comprising a standoff to secure the therapy delivery device to the second portion of the frame.

13. A cannula system for implanting and positioning a medical device through a portal formed in a body, the system comprising:
a frame fixedly attachable to the body relative to the portal;
a guide cannula comprising: a proximal end operable to extend outside the portal; and a distal end implantable within the body, the guide cannula attachable to a first portion of the frame;
an elongate mapping member selectively receivable within the proximal end of the guide cannula, the mapping member having a distal end extendable beyond the distal end of the guide cannula, wherein the mapping member is attachable to a second portion of the frame that is movable relative to the first portion; and
an elongate device placement cannula selectively receivable within the guide cannula and attachable to the second portion, the device placement cannula having a distal end also extendable beyond the distal end of the guide cannula, wherein the device placement cannula has a predetermined effective length that is less than an effective length of the mapping member.

14. A method for positioning a tip of an elongate therapy delivery device in or near a target site within a body, the method comprising:
positioning a guide cannula through a portal formed in the body, wherein a distal end of the guide cannula is located proximate the target site, and a proximal end of the guide cannula is positioned relative to a reference surface located outside the body;
inserting an elongate mapping member into the guide cannula such that a distal end of the mapping member protrudes from the distal end of the guide cannula;
securing a proximal end of the mapping member to a carrier platform that is movable relative to the reference surface, the mapping member having an effective length measured from the carrier platform to the distal end of the mapping member;
determining a location of the target site with the mapping member;
removing the mapping member from the guide cannula;
inserting an elongate device placement cannula into the guide cannula, the placement cannula having an effective length selected to provide a predetermined length differential relative to an effective length of the mapping member;
securing the placement cannula to the carrier platform;
inserting the therapy delivery device into the device placement cannula; and
attaching the therapy delivery device relative to the carrier platform such that a distal end of the therapy delivery device is positioned at or near the target site.

15. The method of claim 14, further comprising selecting the predetermined length differential such that the effective length of the placement cannula is shorter than the effective length of the mapping device.

16. The method of claim 14, wherein determining the location of the target site comprises translating the mapping member relative to the guide cannula and detecting a signal from the mapping device.

17. The method of claim 14, further comprising attaching a standoff to a proximal end of the therapy delivery device, wherein the standoff connects to the carrier platform.

18. The method of claim 14, wherein positioning the guide cannula comprises inserting a first stylet through the guide cannula.

19. The method of claim 14, wherein inserting the device placement cannula into the guide cannula comprises first inserting a second stylet through the device placement cannula.

20. The method of claim 14, further comprising inserting a spacer tube into the guide cannula prior to, or at the time of, inserting the mapping member.

21. The method of claim 14, wherein inserting the mapping member into the guide cannula comprises inserting a microelectrode into the guide cannula.

22. The method of claim 14, wherein the effective length of the device placement cannula is 10 millimeters less than the effective length of the mapping member.

23. The method of claim 14, wherein inserting the therapy delivery device comprises inserting a catheter.
